(19) United States
(12) Patent Application Publication
(10) Pub. No.: US 2026/0061131 A1
(43) Pub. Date: Mar. 5, 2026

(54) SYRINGE FOR AN EYE OPERATION, HAVING A VISCOELASTIC CHAMBER AND A DECOMPOSITION REAGENT CHAMBER

(71) Applicant: Carl Zeiss Meditec AG, Jena (DE)

(72) Inventors: Michael Haisch, Oberkochen (DE); Ben Robertson, Oberkochen (DE)

(21) Appl. No.: 19/384,982

(22) Filed: Nov. 10, 2025

Related U.S. Application Data
(63) Continuation of application No. PCT/EP2024/061230, filed on Apr. 24, 2024.

(30) Foreign Application Priority Data
May 12, 2023 (DE) 10 2023 112 663.6

(51) Int. Cl. A61M 5/19; A61F 9/007

(52) U.S. Cl. CPC A61M 5/19; A61F 9/00736; A61M 2210/0612

(57) ABSTRACT

A syringe is for an eye operation. The syringe has a syringe body, a plunger arranged in the syringe body so as to be displaceable in the syringe longitudinal direction, a viscoelastic chamber in which a viscoelastic is arranged, and a decomposition reagent chamber which is arranged separately from the viscoelastic chamber and in which a decomposition reagent is arranged for decomposing the viscoelastic. The syringe has a storage state, in which the viscoelastic and the decomposition reagent are arranged separately from one another, and a use state, into which the syringe is able to be put by longitudinally displacing the plunger and in which the viscoelastic and the decomposition reagent are mixed with one another.

### CROSS-REFERENCE TO RELATED APPLICATIONS

This application is a continuation application of international patent application PCT/EP2024/061230, filed Apr. 24, 2024, designating the United States and claiming priority from German application 10 2023 112 663.6, filed May 12, 2023, and the entire content of both applications is incorporated herein by reference.

### TECHNICAL FIELD

The disclosure relates to a syringe for an eye operation, wherein the syringe includes a viscoelastic chamber and a decomposition reagent chamber.

### BACKGROUND

In an eye operation, in particular cataract treatment, a viscoelastic is conventionally introduced into the eye. The viscoelastic may, for example, include sodium hyaluronate. The viscoelastic may be a highly viscous cohesive viscoelastic, which is used to maintain a clearance within the eye and to build up pressure, for example in order to dilate the iris of the eye before a capsulorhexis is carried out. The viscoelastic may also be a low-viscosity dispersive viscoelastic, which wets surfaces within the eye to protect these surfaces from injury during the eye operation. The viscoelastic must be removed from the eye at the end of the eye operation because the viscoelastic can block the trabecular meshwork of the eye, which can cause an increase in pressure within the eye. The low-viscosity dispersive viscoelastic wets, in particular, the cornea and the iridocorneal angle of the eye. Because these regions can be reached with an aspiration probe only with difficulty, the low-viscosity dispersive viscoelastic can be removed from the eye only with difficulty. It is known to inject hyaluronidase into the eye. Hyaluronidase is a reagent that decomposes sodium hyaluronate. However, as a result it is necessary during the eye operation to provide a syringe including the hyaluronidase and to introduce the syringe into the eye in order to inject the hyaluronidase. This is a further step to be performed by a physician performing the eye operation, making the eye operation more complex.

### SUMMARY

It is an object of the disclosure to provide a syringe with which an eye operation is less complex.

The syringe according to the disclosure for an eye operation includes a syringe body, a plunger, a viscoelastic chamber and a decomposition reagent chamber. The plunger is arranged in the syringe body so as to be displaceable in a syringe longitudinal direction. A viscoelastic is arranged in the viscoelastic chamber. The decomposition reagent chamber is arranged separately from the viscoelastic chamber and a decomposition reagent configured to decompose the viscoelastic is arranged in the decomposition reagent chamber. The syringe has a storage state, in which the viscoelastic and the decomposition reagent are arranged separately from each other, and a use state, into which the syringe can be brought by a longitudinal displacement of the plunger and in which the viscoelastic and the decomposition reagent are mixed with each other.

By way of the syringe according to the disclosure, it is not necessary to provide a separate syringe for the decomposition reagent, but rather the syringe according to the disclosure provides both the viscoelastic and the decomposition reagent. As a result, the eye operation with the syringe is less complex. In the storage state, mixing of the viscoelastic and the decomposition reagent is avoided, as a result of which a decomposition of the viscoelastic in the storage state is prevented. A mixture of the viscoelastic and the decomposition reagent is only produced when the plunger is longitudinally displaced from the storage state and thus only shortly before the mixture is injected into the eye. This allows the viscoelastic in the eye to perform its function before the viscoelastic is decomposed by the decomposition reagent.

According to various embodiments, It can be preferred for the viscoelastic to include hyaluronic acid and/or a salt of hyaluronic acid and the decomposition reagent to include hyaluronidase and/or for the viscoelastic to include hydroxypropyl methylcellulose and the decomposition reagent to include a cellulase, in particular an exoglucanase and/or an endoglucanase.

According to various embodiments, the syringe can preferably include a cannula, via which the viscoelastic and the decomposition reagent can be displaced out of the syringe by a longitudinal displacement of the plunger in the syringe longitudinal direction. In particular, the mixture of the viscoelastic and the decomposition reagent can be displaced out of the cannula.

According to various embodiments, it can be preferred for the syringe body to include a partition wall, wherein the viscoelastic chamber and the decomposition reagent chamber are arranged next to each other in a direction perpendicular to the syringe longitudinal direction and are separated from each other via the partition wall. It is particularly preferred for the syringe to include a mixing chamber, which is arranged such that the viscoelastic and the decomposition reagent enter the mixing chamber when the plunger is displaced in the syringe longitudinal direction. In the mixing chamber, the viscoelastic and the decomposition reagent can mix and thus form the mixture. It is conceivable that, in the storage state, the mixing chamber is free of the viscoelastic and/or the decomposition reagent. This can for example be achieved in that the syringe includes a viscoelastic closure via which a flow of the viscoelastic into the mixing chamber is prevented when the syringe is in the storage state, and which can be opened by displacing the plunger in the syringe longitudinal direction from the storage state, and/or in that the syringe includes a decomposition reagent closure via which a flow of the decomposition reagent into the mixing chamber is prevented when the syringe is in the storage state, and which can be opened by displacing the plunger in the syringe longitudinal direction from the storage state. As an alternative or in addition, it is conceivable for the in the storage state air or another gas to be arranged in the mixing chamber, which prevents the entry of the viscoelastic and/or the decomposition reagent into the mixing chamber.

According to various embodiments, the it is preferred for the mixing chamber to be arranged such that the viscoelastic and the decomposition reagent together exit the mixing chamber and then pass into the cannula when the plunger is displaced in the syringe longitudinal direction. Thus, the mixing chamber lies downstream of the viscoelastic chamber, downstream of the decomposition reagent chamber and upstream of the cannula in relation to a flow direction brought about by the plunger being displaced in the syringe longitudinal direction.

As an alternative to providing the mixing chamber, it is conceivable for the viscoelastic and the decomposition reagent together to enter the cannula and there the mixture is formed. In another example in which the mixing chamber is also not provided, it is conceivable for the cannula to include a first line which the viscoelastic enters when the plunger is displaced in the syringe longitudinal direction, and a second line which the decomposition reagent enters when the plunger is displaced in the syringe longitudinal direction, wherein the cannula is configured such that the mixture only forms after the viscoelastic and the decomposition reagent have exited the cannula.

According to various embodiments, the syringe preferably has a tip region and a body region, wherein the tip region can be detachably coupled with the body region, in particular via a thread or by mounting the tip region on the body region in a longitudinally displaceable manner, wherein the mixing chamber is arranged in the tip region and the viscoelastic chamber and the decomposition reagent chamber are arranged in the body region.

According to various embodiments, the plunger preferably includes a first partial plunger, which is configured to displace the viscoelastic out of the viscoelastic chamber, and a second partial plunger, which is arranged separately from the first partial plunger and is configured to displace the decomposition reagent out of the decomposition reagent chamber. It is particularly preferred for the second partial plunger to have a predetermined breaking point. By breaking the second partial plunger at the predetermined breaking point, it is possible to remove the broken-off part of the second partial plunger from the injector body. If the plunger is then displaced in the syringe longitudinal direction, only the viscoelastic is displaced out of the cannula.

According to various embodiments, the syringe includes a stopper, which is arranged in the injector body so as to be displaceable in the syringe longitudinal direction and can be driven from outside the injector body for this purpose, wherein the decomposition reagent chamber and the viscoelastic chamber are arranged consecutively in the syringe longitudinal direction. In other words, the viscoelastic chamber is arranged downstream of the decomposition reagent chamber in relation to a flow direction brought about by the plunger being displaced in the syringe longitudinal direction. Furthermore, the viscoelastic chamber and the decomposition reagent chamber are separated from each other via the stopper, which delimits a line via which the viscoelastic can flow from the viscoelastic chamber into the decomposition reagent chamber and the decomposition reagent can flow from the decomposition reagent chamber into the viscoelastic chamber. By displacing the stopper in the syringe longitudinal direction, the viscoelastic or the decomposition reagent flows counter to the syringe longitudinal direction in the line and, after exiting the line, mixes with the decomposition reagent or the viscoelastic there. By displacing the stopper counter to the syringe longitudinal direction, the viscoelastic or the decomposition reagent flows in the syringe longitudinal direction in the line and, after exiting the line, mixes with the decomposition reagent or the viscoelastic there. By displacing the stopper in the syringe longitudinal direction and counter to the syringe longitudinal direction several times, the viscoelastic and the decomposition reagent can be mixed particularly well.

According to various embodiments, the stopper preferably includes a valve, which is configured to block the line in the storage state and to open the line when a pressure difference between the viscoelastic chamber and the decomposition reagent chamber is exceeded. This can prevent the viscoelastic and the decomposition reagent from mixing already in the storage state.

According to various embodiments, it is preferred for the plunger to be formed by an outer plunger and an inner plunger which is mounted longitudinally displaceably within the outer plunger, wherein the stopper is either fastened to the outer plunger or to the inner plunger. As a result, the stopper can be driven from outside the injector body in the syringe longitudinal direction and counter to the syringe longitudinal direction in a particularly simple manner.

According to various embodiments, the viscoelastic chamber is preferably arranged in the syringe longitudinal direction between the cannula and the decomposition reagent chamber.

### BRIEF DESCRIPTION OF DRAWINGS

The invention will now be described with reference to the drawings wherein:

FIG. 1 shows a longitudinal section through a first embodiment of the syringe, having a first embodiment of a viscoelastic chamber and a decomposition reagent chamber of the syringe,

FIG. 2 shows a perspective view of a second embodiment of the viscoelastic chamber and the decomposition reagent chamber,

FIG. 3 shows a perspective view of a third embodiment of the viscoelastic chamber and the decomposition reagent chamber,

FIG. 4 shows a perspective view of the first embodiment of the viscoelastic chamber and the decomposition reagent chamber, and

FIG. 5 shows a longitudinal section through a second embodiment of the syringe.

### DETAILED DESCRIPTION

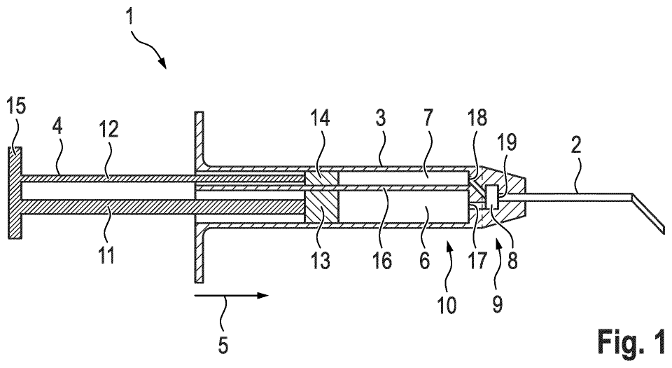

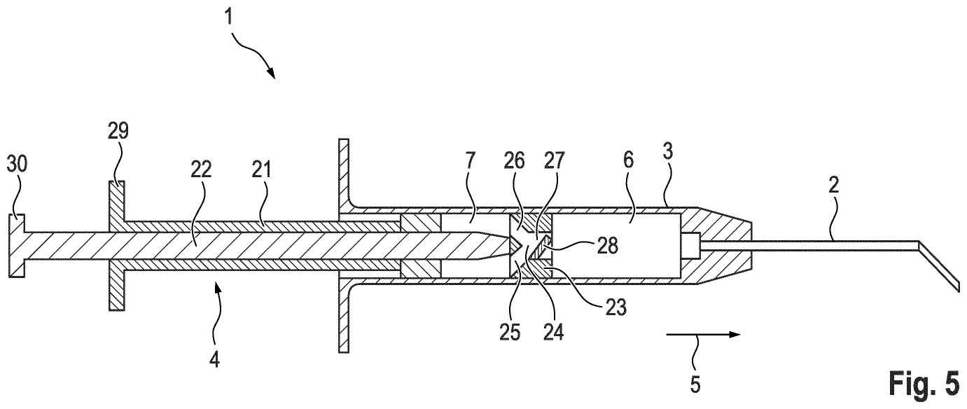

As can be seen from FIGS. 1 and 5, a syringe 1 for an eye operation includes a syringe body 3, a plunger 4, a viscoelastic chamber 6 and a decomposition reagent chamber 7. The plunger 4 is arranged in the syringe body 3 so as to be displaceable in a syringe longitudinal direction 5. A viscoelastic is arranged in the viscoelastic chamber 6. The decomposition reagent chamber 7 is arranged separately from the viscoelastic chamber 6, wherein a decomposition reagent configured to decompose the viscoelastic is arranged in the decomposition reagent chamber 7. The syringe 1 has a storage state, in which the viscoelastic and the decomposition reagent are arranged separately from each other and in particular in their respective chamber 6, 7, and a use state, into which the syringe 1 can be brought by a longitudinal displacement of the plunger 4 and in which the viscoelastic and the decomposition reagent are mixed with each other. By displacing the plunger 4 in the syringe longitudinal direction 5 from the storage state, a mixture can be produced within the syringe 1, the mixture including the viscoelastic and the decomposition reagent and in particular consisting of the viscoelastic and the decomposition reagent. In particular, the syringe 1 may be configured such that, in the storage state, no mixing of the viscoelastic and the decomposition reagent takes place. The syringe 1 may be configured to then inject the mixture into the eye. The term “use state” refers to the syringe in any state of the syringe in which plunger 4 has been displaced in the syringe longitudinal direction 5 from the storage state.

The viscoelastic may include hyaluronic acid and/or a salt of hyaluronic acid and the decomposition reagent may include hyaluronidase and/or the viscoelastic may include hydroxypropyl methylcellulose and the decomposition reagent may include a cellulase, in particular an exoglucanase and/or an endoglucanase. The hyaluronidase is configured to decompose the hyaluronic acid and/or the salt of hyaluronic acid. The cellulase is configured to decompose the hydroxypropyl methylcellulose. The viscoelastic may be free of the decomposition reagent and the decomposition reagent may be free of the viscoelastic.

It is conceivable for the viscoelastic to be present in liquid form. In a first exemplary solution which forms the viscoelastic, for this purpose the hyaluronic acid and/or the salt of hyaluronic acid may be dissolved in a solvent, such as water. The salt of hyaluronic acid may, for example, be sodium hyaluronate. The sodium hyaluronate may, for example, have an average molar mass in a range from 0.5 to 4 MDa, in particular in a range from 0.5 to 1.5 MDa. The concentration of the sodium hyaluronate in the first solution may, for example, lie in a range from 0.5% by mass to 4.0% by mass, in particular in a range from 2.0% by mass to 4.0% by mass. The viscoelastic may additionally include auxiliary substances, such as a pH buffer, in particular a phosphate buffer, NaCl, lidocaine, NaOH, sodium hydrogen carbonate and/or antioxidants. In a second exemplary solution which forms the decomposition reagent, the hydroxypropyl methylcellulose may be dissolved in a solvent, such as water. The hydroxypropyl methylcellulose may, for example, have an average molar mass in a range from 10 kDa to 2000 kDa, in particular in a range from 50 kDa to 500 kDa. The concentration of the hydroxypropyl methylcellulose in the viscoelastic may, for example, lie in a range from 0.5% by mass to 4.0% by mass, in particular in a range from 1.0% by mass to 3.0% by mass. The viscoelastic may additionally include auxiliary substances, such as a pH buffer, in particular a phosphate buffer, NaCl, lidocaine, NaOH, sodium hydrogen carbonate and/or antioxidants.

It is conceivable for the decomposition reagent to be present in liquid form. For example, for this purpose, in a third exemplary solution which forms the decomposition reagent, the hyaluronidase may be dissolved in a solvent, such as water. The concentration of the hyaluronidase in the third solution may lie in a range from 0.1 to 10 U/ml, in particular in a range from 0.5 to 2 U/ml. The decomposition reagent may additionally include auxiliary substances, such as a pH buffer, in particular a phosphate buffer. In a fourth exemplary solution which forms the decomposition reagent, the cellulase may be dissolved in a solvent, such as water. The concentration of the cellulase in the decomposition reagent may lie in a range from 0.1 to 20 U/ml, in particular in a range from 0.5 to 5 U/ml. The decomposition reagent may additionally include auxiliary substances, such as a pH buffer, in particular a phosphate buffer.

It is also conceivable for the decomposition reagent to be a mixture of the first solution and the second solution. The volume ratio of the first solution to the second solution may, for example, lie in a range from 1:100 to 100:1, in particular in a range from 1:10 to 10:1. In the case that the decomposition reagent is the mixture of the first solution and the second solution, it is conceivable for the decomposition reagent to be a mixture of the third solution and the fourth solution. The volume ratio of the third solution to the fourth solution may, for example, lie in a range from 1:100 to 100:1, in particular in a range from 1:10 to 10:1.

The volume ratio of viscoelastic to decomposition reagent in the syringe 1 may be set in such a way that an increase in the viscosity in the eye brought about by the injection of the viscoelastic is reduced by 15% to 60%, in particular by 25% to 50%, within 5 to 15 minutes.

FIGS. 1 and 5 show that the syringe 1 may include a cannula 2, via which the viscoelastic and the decomposition reagent can be displaced out of the syringe 1 by a longitudinal displacement of the plunger 4 in the syringe longitudinal direction 5. In a flow direction of the viscoelastic and/or the decomposition reagent brought about by a displacement of the plunger 4 in the syringe longitudinal direction 5, the cannula 2 is arranged downstream of the viscoelastic chamber 6 and downstream of the decomposition reagent chamber 7.

According to a first embodiment of the syringe 1 illustrated in FIGS. 1 and 4, the syringe body 3 may include a partition wall 16, wherein the viscoelastic chamber 6 and the decomposition reagent chamber 7 are arranged next to each other in a direction perpendicular to the syringe longitudinal direction 5 and are separated from each other via the partition wall 16.

FIG. 1 shows that the syringe 1 may include a mixing chamber 8, which is arranged such that the viscoelastic and the decomposition reagent enter the mixing chamber 8 when the plunger 4 is displaced in the syringe longitudinal direction 5. In the mixing chamber 8, the mixture can thus be formed. In addition, the mixing chamber 8 may be arranged such that the viscoelastic and the decomposition reagent together exit the mixing chamber 8 and then pass into the cannula 2 when the plunger 4 is displaced in the syringe longitudinal direction 5, that is, the mixing chamber 8 is arranged downstream of the viscoelastic chamber 6, downstream of the decomposition reagent chamber 7 and upstream of the cannula 2 in relation to the flow direction. The mixing chamber 8 may include a first mixing chamber opening 17, via which the mixing chamber 8 is fluid-conductingly connected to the viscoelastic chamber 6 in the use state. The mixing chamber 8 may include a second mixing chamber opening 18, via which the mixing chamber 8 is fluid-conductingly connected to the decomposition reagent chamber 7 in the use state. It is conceivable for the first mixing chamber opening 17 to be arranged spaced apart from the second mixing chamber opening 18. The mixing chamber 8 may include a third mixing chamber opening 19, via which the mixing chamber 8 is fluid-conductingly connected to the cannula 2. The third mixing chamber opening 19 may be arranged spaced apart from the first mixing chamber opening 17 and the second mixing chamber opening 18.

It is conceivable for the syringe 1 to include a viscoelastic closure via which a flow of the viscoelastic from the viscoelastic chamber 6 into the mixing chamber 8 is prevented when the syringe 1 is in the storage state, wherein the viscoelastic closure can be opened by displacing the plunger 4 in the syringe longitudinal direction 5 from the storage state. This may be achieved in that, in the storage state, no mixing of the viscoelastic and the decomposition reagent takes place. As an alternative or in addition, it is conceivable for the syringe 1 to include a decomposition reagent closure via which a flow of the decomposition reagent from the decomposition reagent chamber 7 into the mixing chamber 8 is prevented when the syringe 1 is in the storage state, wherein the decomposition reagent closure can be opened by displacing the plunger 4 in the syringe longitudinal direction 5 from the storage state.

The syringe 1 may have a tip region 9 and a body region 10, wherein the tip region 9 can be detachably coupled with the body region 10, wherein the mixing chamber 8 is arranged in the tip region 9 and the viscoelastic chamber 6 and the decomposition reagent chamber 7 are arranged in the body region 10. In FIG. 1, the dashed line indicates the boundary between the tip region 9 and the body region 10. In order to detachably couple the tip region 9 with the body region 10, the syringe 1 may, for example, have a thread or the tip region 9 may be arranged on the body region 10 in a longitudinally displaceable manner.

It is conceivable for the cross section of the decomposition reagent chamber 7 in a plane whose normal is arranged parallel to the syringe longitudinal direction 5 to be smaller than the cross section of the viscoelastic chamber 6 in the plane. This allows the decomposition reagent to be provided with a smaller volume than the viscoelastic. In particular, the cross section of the decomposition reagent chamber 7 is less than or equal to 50% of the cross section of the viscoelastic chamber 6.

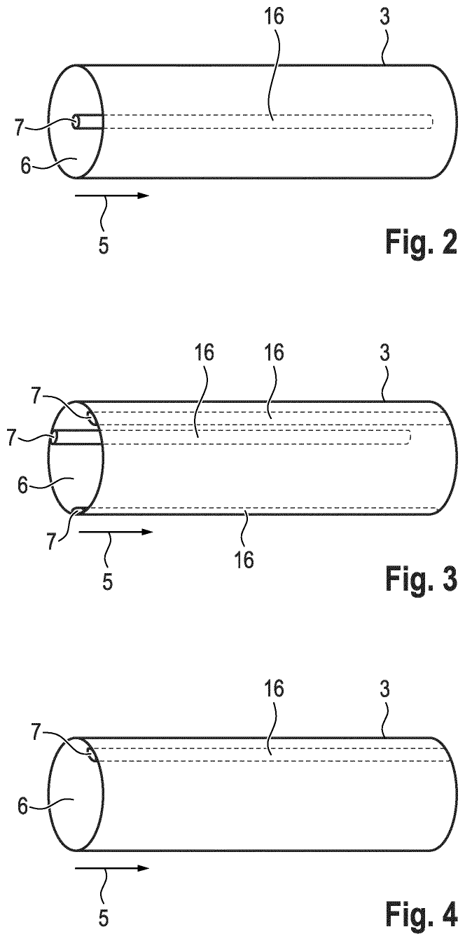

As can be seen from FIGS. 1 and 2, the plunger 4 may include a first partial plunger 11, which is configured to displace the viscoelastic out of the viscoelastic chamber 6, and a second partial plunger 12, which is arranged separately from the first partial plunger 11 and is configured to displace the decomposition reagent out of the decomposition reagent chamber 7. In this case, the first partial plunger 11 may delimit the viscoelastic chamber 6 and the second partial plunger 12 may delimit the decomposition reagent chamber 7. It is conceivable for the plunger 4 to include a thumb rest 15, which forms the end of the plunger 4 facing away from the viscoelastic chamber 6. The first partial plunger 11 and the second partial plunger 12 may be fastened to the thumb rest 15. As a result, the first partial plunger 11 and the second partial plunger 12 can be displaced simultaneously by pressing of the thumb rest 15. It is conceivable for the first partial plunger 11 to have a first plunger thickening 13, which forms the end of the first partial plunger 11 facing away from the thumb rest 15. In addition, it is conceivable for the second partial plunger 12 to have a second plunger thickening 14, which forms the end of the second partial plunger 12 facing away from the thumb rest 15.

It is conceivable for the second partial plunger 12 to have a predetermined breaking point. The second partial plunger 12 can be broken at the predetermined breaking point and a portion of the second partial plunger 12, the portion including the longitudinal end of the second partial plunger 12 facing away from the thumb rest 15, can be removed from the injector body 2. As an alternative to providing the predetermined breaking point, it is also conceivable for the second partial plunger 12 to not be fastened indirectly or directly to the thumb rest 15, but rather only the first partial plunger 12 to be fastened to the thumb rest 15, as a result of which the second partial plunger 12 can be removed from the injector body 2. It is also conceivable for the thumb rest 15 to include a through hole through which the second partial plunger 12 extends. In this case, the second partial plunger 12 may include a locking mechanism which has an unlocked state, in which the second partial plunger 12 can slide through the through hole, and a locked state, in which the second partial plunger 12 is fastened to the thumb rest 15. In the locked state, only the viscoelastic is pressed out of the syringe 1 by a longitudinal displacement of the plunger 4, whereas in the unlocked state, the viscoelastic and the decomposition reagent are pressed out of the syringe 1 by a longitudinal displacement of the plunger 4. This allows for example first the viscoelastic alone and later the viscoelastic together with the decomposition reagent to be injected. For example, the locking mechanism may be formed by teeth on the second partial plunger 12, which are not in engagement with the thumb rest 15 in the unlocked state and are in engagement with the thumb rest 15 in the locked state. For example, the teeth may be brought into engagement by rotation of the second partial plunger 12.

FIGS. 1 and 4 show a first embodiment of the viscoelastic chamber 6 and the decomposition reagent chamber 7, in which the decomposition reagent chamber 7 is not enveloped by the viscoelastic chamber 6, but rather is arranged completely laterally to the viscoelastic chamber 6 in a direction transverse to the syringe longitudinal direction 5. FIG. 2 shows a second embodiment of the viscoelastic chamber 6 and the decomposition reagent chamber 7, in which the viscoelastic chamber 6 envelops the decomposition reagent chamber 7. FIG. 3 shows a third embodiment of the viscoelastic chamber 6 and the decomposition reagent chamber 7, in which a plurality of decomposition reagent chambers 7 are provided. It is conceivable for a respective one of the second partial plungers 12 to be provided for each of the decomposition reagent chambers 7.

According to a second embodiment of the syringe 1 illustrated in FIG. 5, the syringe 1 may include a stopper 23, which is arranged in the injector body 3 so as to be displaceable in the syringe longitudinal direction 5 and can be driven from outside the injector body 3 for this purpose, wherein the decomposition reagent chamber 7 and the viscoelastic chamber 6 are arranged consecutively in the syringe longitudinal direction 5. In other words, the viscoelastic chamber 6 is arranged downstream of the decomposition reagent chamber 7 in relation to a flow direction brought about by the plunger 4 being displaced in the syringe longitudinal direction 5. In addition, the viscoelastic chamber 6 and the decomposition reagent chamber 7 are separated from each other via the stopper 23, which delimits a line 24 via which the viscoelastic can flow from the viscoelastic chamber 6 into the decomposition reagent chamber 7 and the decomposition reagent can flow from the decomposition reagent chamber 7 into the viscoelastic chamber 6. The stopper 23 may include a valve 28, which is configured to block the line 24 in the storage state and to open the line 24 when a pressure difference between the viscoelastic chamber 6 and the decomposition reagent chamber 7 is exceeded. As a result, the syringe 1 is configured such that, in the storage state, no mixing of the viscoelastic and the decomposition reagent takes place. If the pressure in the viscoelastic chamber 6 is higher than the pressure in the decomposition reagent chamber 7 by a first pressure difference, the valve 28 opens the line 24. If the pressure in the decomposition reagent chamber 7 is higher than the pressure in the viscoelastic chamber 6 by a second pressure difference, the valve 28 opens the line 24. It is conceivable for the first pressure difference to be equal to the second pressure difference. As an alternative, it is also conceivable for the valve 28 to be a film which tears when a pressure difference between the viscoelastic chamber 6 and the decomposition reagent chamber 7 is exceeded.

FIG. 5 shows that the plunger 4 may be formed by an outer plunger 21 and an inner plunger 22 which is mounted longitudinally displaceably within the outer plunger 21, wherein the stopper 23 is fastened to the inner plunger 22. As an alternative, it is conceivable for the stopper 23 to be fastened to the outer plunger 21. By fastening the stopper 23 to the inner plunger 22 or to the outer plunger 23, the stopper 23 can be driven from outside the injector body 2 in the syringe longitudinal direction 5 or counter to the syringe longitudinal direction 5. It is conceivable for the outer plunger 21 to include an outer plunger thumb rest 29, which is a thickening of the outer plunger 21 at the longitudinal end of the outer plunger 21 facing away from the cannula 2. In addition, the inner plunger 22 may include an inner plunger thumb rest 30, which is a thickening of the inner plunger 22 at the longitudinal end of the inner plunger 22 facing away from the cannula 2 and which is arranged outside the outer plunger 21 in the storage state and in the use state.

The line 24 may include a first sub-line 25 and a second sub-line 26 which communicate with the one of the viscoelastic chamber 6 and the decomposition reagent chamber 7 which is arranged on the side of the stopper 23 facing away from the cannula 2. The first sub-line 25 and the second sub-line 26 may open, in a direction perpendicular to the syringe longitudinal direction 5, on different sides of the inner plunger 22 into the one of the viscoelastic chamber 6 and the decomposition reagent chamber 7 which is arranged on the side of the stopper 23 facing away from the cannula 2 (see FIG. 5). In addition, the line 24 may include a third sub-line 27 which opens into the one of the viscoelastic chamber 6 and the decomposition reagent chamber 7 which opens on the side of the stopper 23 facing the cannula 2, wherein the first sub-line 25 and the second sub-line 26 open into the third sub-line 27. The valve 28 may be arranged in the third sub-line 27.

The viscoelastic chamber 6 may be arranged in the syringe longitudinal direction 5 between the cannula 2 and the decomposition reagent chamber 7 (see FIG. 5).

By then displacing the stopper 23 via the inner plunger 22 in the syringe longitudinal direction 5, the viscoelastic flows counter to the syringe longitudinal direction 5 through the line 24 into the decomposition reagent chamber 7 and mixes with the decomposition reagent there. It is conceivable for the outer plunger 21 to displace counter to the syringe longitudinal direction 5 in order to compensate for the volume of the inner plunger 21 in the viscoelastic chamber 7. If the stopper 23 is then displaced via the inner plunger 22 counter to the syringe longitudinal direction 5, the mixture of the viscoelastic and the decomposition reagent flows in the syringe longitudinal direction 5 through the line 24 into the viscoelastic chamber 6. At the same time, the outer plunger 21 can displace in the syringe longitudinal direction 5 in order to compensate for the volume of the inner plunger 22 in the decomposition reagent chamber 7.

It is understood that the foregoing description is that of the preferred embodiments of the invention and that various changes and modifications may be made thereto without departing from the spirit and scope of the invention as defined in the appended claims.

### LIST OF REFERENCE SIGNS

1 Syringe
2 Cannula
3 Syringe body
4 Plunger
Syringe longitudinal direction
6 Viscoelastic chamber
7 Decomposition reagent chamber
8 Mixing chamber
9 Tip region
Body region
11 First partial plunger
12 Second partial plunger
13 First plunger thickening
14 Second plunger thickening
Thumb rest
16 Partition wall
17 First mixing chamber opening
18 Second mixing chamber opening
19 Third mixing chamber opening
21 Outer plunger
22 Inner plunger
23 Stopper
24 Line
25 First sub-line
26 Second sub-line
27 Third sub-line
28 Valve
29 Outer plunger thumb rest
30 Inner plunger thumb rest

1. A syringe for an eye operation, the syringe comprising:
a syringe body;
a plunger arranged in said syringe body so as to be displaceable in a syringe longitudinal direction;
a viscoelastic chamber in which a viscoelastic is arranged;
a decomposition reagent chamber arranged separately from said viscoelastic chamber and in which a decomposition reagent configured to decompose the viscoelastic is arranged; and,
wherein the syringe has a storage state, in which the viscoelastic and the decomposition reagent are arranged separately from each other, and a use state, into which the syringe can be brought by a longitudinal displacement of said plunger and in which the viscoelastic and the decomposition reagent are mixed with each other.

2. The syringe of claim 1, wherein at least one of:
the viscoelastic includes at least one of hyaluronic acid and a salt of hyaluronic acid and the decomposition reagent includes hyaluronidase; and,
the viscoelastic includes hydroxypropyl methylcellulose and the decomposition reagent includes a cellulase.

3. The syringe of claim 1 further comprising a cannula via which the viscoelastic and the decomposition reagent are displaceable out of the syringe by a longitudinal displacement of said plunger in the syringe longitudinal direction.

4. The syringe of claim 3, wherein said syringe body includes a partition wall; and, said viscoelastic chamber and said decomposition reagent chamber are arranged next to each other in a direction perpendicular to the syringe longitudinal direction and are separated from each other via said partition wall.

5. The syringe of claim 4 further comprising:
a mixing chamber arranged such that the viscoelastic and the decomposition reagent enter said mixing chamber when said plunger is displaced in the syringe longitudinal direction.

6. The syringe of claim 5, wherein said mixing chamber is arranged such that the viscoelastic and the decomposition reagent together exit said mixing chamber and then pass into said cannula when said plunger is displaced in the syringe longitudinal direction.

7. The syringe of claim 5, wherein the syringe has a tip region and a body region; said tip region is configured to be detachably coupled with said body region; and, said mixing chamber is arranged in said tip region and said viscoelastic chamber and said decomposition reagent chamber are arranged in said body region.

8. The syringe of claim 4, wherein said plunger includes a first partial plunger configured to displace the viscoelastic out of said viscoelastic chamber and a second partial plunger arranged separately from said first partial plunger; and, said second partial plunger is configured to displace the decomposition reagent out of said decomposition reagent chamber.

9. The syringe of claim 8, wherein said second partial plunger has a predetermined breaking point.

10. The syringe of claim 3 further comprising:
a stopper arranged in said syringe body so as to be displaceable in the syringe longitudinal direction and configured to be driven from outside said syringe body for this displacement; and,
said decomposition reagent chamber and said viscoelastic chamber being arranged consecutively in the syringe longitudinal direction and being separated from each other via said stopper, which delimits a line via which the viscoelastic can flow from said viscoelastic chamber into said decomposition reagent chamber and the decomposition reagent can flow from said decomposition reagent chamber into said viscoelastic chamber.

11. The syringe of claim 10, wherein said stopper includes a valve configured to block said line in the storage state and to open said line when a pressure difference between said viscoelastic chamber and said decomposition reagent chamber is exceeded.

12. The syringe of claim 10, wherein said plunger is formed by an outer plunger and an inner plunger which is mounted longitudinally displaceably within said outer plunger; and, said stopper is either fastened to said outer plunger or to said inner plunger.

13. The syringe of claim 1, wherein the viscoelastic includes hydroxypropyl methylcellulose and the decomposition reagent includes at least one of an exoglucanase and an endoglucanase.
